(19) United States
(12) Patent Application Publication
(10) Pub. No.: US 2026/0172935 A1
(43) Pub. Date: Jun. 18, 2026

(54) Time Division Management for Single-Antenna Communications in Ambulatory Infusion Systems

(71) Applicant: Tandem Diabetes Care, Inc., San Diego, CA (US)

(72) Inventors: Paul Harris, San Diego, CA (US); Angelo Rodriguez, Riverside, CA (US); Dwight Lee, Encinitas, CA (US); Chris Merz, Carlsbad, CA (US); Kuni Tran, San Diego, CA (US); Sydney Choi, San Diego, CA (US)

(21) Appl. No.: 19/417,074

(22) Filed: Dec. 11, 2025

Related U.S. Application Data
(60) Provisional application No. 63/733,448, filed on Dec. 13, 2024.

(51) Int. Cl. H04W 36/26; A61M 5/142; A61M 5/172; H03K 3/017

(52) U.S. Cl. CPC H04W 36/26; A61M 5/14244; A61M 5/1723; H03K 3/017; A61M 2205/3561; A61M 2205/3584; A61M 2230/201

(57) ABSTRACT

The present disclosure regards an electronic device for insulin delivery management. The electronic device includes an antenna and a processor. The processor is configured to operate in a first communication mode and then operate in a second communication mode responsive to determining that one or more communication criteria are satisfied. The first communication mode involves communicating via the antenna with first and second peripheral devices according to respective first and duty cycles. And the second communication mode involves communicating via the antenna with the first and second peripheral devices according to respective first and second adjusted duty cycles. The first and second duty cycles are different from the first and second adjusted duty cycles, respectively.

### PRIORITY

This application claims the benefit of U.S. Provisional App. No. 63/733,448 (filed Dec. 13, 2024), the entirety of which is incorporated herein by reference.

### TECHNICAL FIELD

The present disclosure relates, generally, to wireless communication technologies and, more specifically, to time division management for wireless communications between host and peripheral devices.

### BACKGROUND

Many wireless communication standards allow only for communication between two separate devices at any given time. For instance, Bluetooth operates on a host-peripheral architecture that fundamentally limits the ability of a host device to communicate simultaneously with multiple peripheral devices. Other wireless standards have similar limitations, including Zigbee, IrDA, and ANT.

In practice, this means that while a host device is communicating with a peripheral device, other connected peripheral devices must wait for the ongoing communication to end before the other peripheral devices can transmit or receive their own data. This lack of simultaneous communication often results in increased latency and reduced efficiency—particularly in scenarios where real-time data exchange is crucial.

### SUMMARY

The present disclosure seeks to improve communication in multi-device systems where one or more devices in the system (e.g., a host device) are incapable of, or at least ill-suited for, simultaneous communication. To that end, many of the devices, systems, and methods discussed herein involve a host device configured to operate in accordance with various communication modes, where each mode includes respective duty cycles for each of a plurality of peripheral devices connected to the host device. The communication modes are tailored for particular circumstances in order to improve the efficiency of communication between the host and peripheral devices under said circumstances.

As used herein, “duty cycle” refers to the amount of time a host device dedicates to communicating with a given peripheral device for a period of time. For instance, consider a host device with two connected peripherals. If the host device spends six out of every ten seconds communicating with the first peripheral device, then the host device can be said to communicate with the first peripheral device according to a 60% duty cycle. Similarly, if the host device spends four out of every ten seconds communicating with the second peripheral device, then the host device is communicating with that device according to a 40% duty cycle.

Exemplary embodiments include the following:

An Electronic Device. The electronic device is configured for insulin delivery management, and it includes an antenna and a processor. The processor is configured to operate in a first communication mode. The first communication mode involves communicating via the antenna with a first peripheral device according to a first duty cycle and a second peripheral device according to a second duty cycle. The processor is also configured to determine that one or more communication criteria are satisfied while operating in the first communication mode. Additionally, the processor is configured to and responsive to determining that the one or more communication criteria are satisfied, switch from operating in the first communication mode to operating in a second communication mode. The second communication mode involves communicating via the antenna with the first peripheral device according to a first adjusted duty cycle different from the first duty cycle and the second peripheral device according to a second adjusted duty cycle different from the second duty cycle.

A Computer-Implemented Method. The method is a method of managing time division for single-antenna communications regarding insulin delivery management. It includes operating a processor of an electronic device in a first communication mode. The first communication mode involves communicating via an antenna of the electronic device with a first peripheral device according to a first duty cycle and a second peripheral device according to a second duty cycle. The method also includes, while operating the processor in the first communication mode, determining that one or more communication criteria are satisfied. Additionally, the method includes, responsive to determining that the one or more communication criteria are satisfied, switching from operating the processor in the first communication mode to operating the processor in a second communication mode. The second communication mode involves communicating via the antenna with the first peripheral device according to a first adjusted duty cycle different from the first duty cycle and the second peripheral device according to a second adjusted duty cycle different from the second duty cycle.

A Non-Transitory, Computer-Readable Medium. The computer-readable medium includes instructions that, when executed by a processor of an electronic device for insulin delivery management, cause the electronic device to operate in a first communication mode. The first communication mode involves communicating via an antenna of the electronic device with a first peripheral device according to a first duty cycle and a second peripheral device according to a second duty cycle. The instructions also cause the electronic device to, while operating in the first communication mode, determine that one or more communication criteria are satisfied. Additionally, the instructions cause the electronic device to, responsive to determining that the one or more communication criteria are satisfied, switch from operating in the first communication mode to operating in a second communication mode. The second communication mode involves communicating via the antenna with the first peripheral device according to a first adjusted duty cycle different from the first duty cycle and the second peripheral device according to a second adjusted duty cycle different from the second duty cycle.

A System. The system is for insulin delivery management, and it includes a first peripheral device, a second peripheral device, and an electronic device. The electronic device includes an antenna and a processor. The processor is configured to operate in a first communication mode. The first communication mode involves communicating via the antenna with the first peripheral device according to a first duty cycle and the second peripheral device according to a second duty cycle. The processor is also configured to, while operating in the first communication mode, determine that one or more communication criteria are satisfied. Additionally, the processor is configured to, responsive to determining that the one or more communication criteria are satisfied, switch from operating in the first communication mode to operating in a second communication mode. The second communication mode involves communicating via the antenna with the first peripheral device according to a first adjusted duty cycle different from the first duty cycle and the second peripheral device according to a second adjusted duty cycle different from the second duty cycle.

Based on the following Detailed Description, other configurations of the subject technology will be apparent to those skilled in the art. The Detailed Description describes various configurations of the subject technology, particularly with respect to illustrations thereof. Notwithstanding, the subject technology is capable of other and different configurations, and its several details are capable of modification in various other respects-all without departing from the scope of the subject technology. The Drawings and Detailed Description are therefore presented as illustrative in nature and should not be construed as restricting the present disclosure.

### BRIEF DESCRIPTION OF THE DRAWINGS

For a better understanding of the present disclosure, reference should be made to the Detailed Description, below, in conjunction with the following drawings. Like reference numerals refer to corresponding parts throughout the figures and the description.

FIGS. 1A through 1C illustrate example systems for wireless communication between a host device and two peripheral devices, according to various aspects of the subject technology.

FIGS. 2A through 2C illustrate example communication modes and corresponding duty cycles for communication between a host device and multiple peripheral devices, according to various aspects of the subject technology.

FIGS. 3A through 3D illustrate example communication scenarios involving an ambulatory infusion pump and peripheral devices connected thereto, according to various aspects of the subject technology.

FIG. 4 illustrates an example process for managing time division for single-antenna communications between a host device and multiple peripheral devices, according to various aspects of the subject technology.

### DETAILED DESCRIPTION

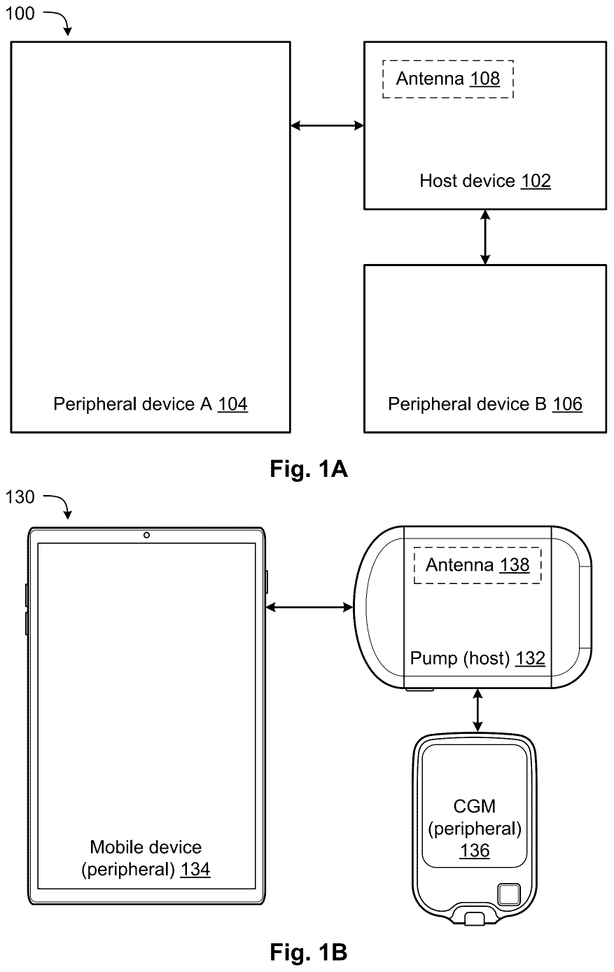

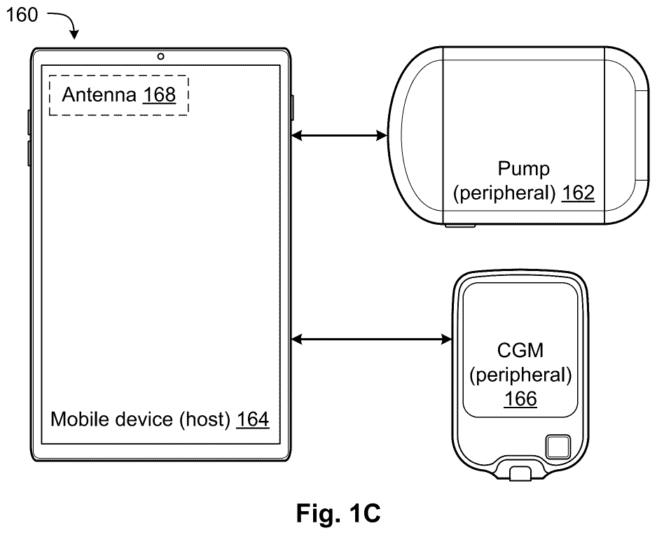

FIGS. 1A and 1B illustrate example systems 100, 130 and 160 for wireless communication between a host device and two peripheral devices, according to various aspects of the subject technology.

In FIG. 1A, the system 100 includes a generic host device 102, as well as generic peripheral devices 104 and 106. The host device 102 is configured to communicate with the peripheral devices 104 and 106 via an antenna 108; however, the host device 102 is incapable of, or at least ill-suited for, communicating with the peripheral devices 104 and 106 at the same time. For instance, the host device may lack the hardware necessary for simultaneous communication (e.g., an additional antenna), or it may use a wireless communication protocol that precludes such communication (e.g., Bluetooth).

The peripheral devices 104 and 106 are configured to receive commands from the host device 102 and handle the commands accordingly. These devices 104 and 106 can include more common peripherals, such as headphones, mice and keyboards, external memory drives, printers, and the like. Nonetheless, more specialized peripheral devices are also within the scope of the present disclosure. Much of the following discussion, for instance, involves devices for use in ambulatory infusion systems (e.g., mobile devices, infusion pumps, glucose monitors).

FIG. 1B illustrates such a system, with an ambulatory infusion pump 132 connected to, and configured to communicate with, a mobile device 134 (e.g., a tablet, a smartphone) and a continuous glucose monitor (CGM) 136. As with the first system 100, communication in this system 130 occurs via an antenna 138 of the host device (i.e., the pump 132) and with only one peripheral device (e.g., mobile device 134, CGM 136) at a time. Accordingly, the ambulatory infusion pump 132 is unable to receive data from the CGM 136 while also communicating with the mobile device 134, and vice versa.

This limitation can be particularly problematic in multi-device systems involving the transmission of real-time data or time-sensitive requests. In the second system 130, for example, the CGM 136 can be configured to collect blood glucose data for a patient and transmit that data to the ambulatory infusion pump 132 at regular intervals. The pump 132 can then use this data to make real-time adjustments to the amount of insulin it provides to its user. Meanwhile, the mobile device 134 can be configured to send various requests (e.g., a bolus request, a basal rate adjustment request) to the pump 132, many of which may require its near-immediate attention.

Accordingly, in various embodiments of the subject technology, the ambulatory infusion pump 132 is configured to operate in two or more communication modes—many of which prioritize communication with one peripheral device over another. For instance, if the pump 132 receives a bolus request from the mobile device 134, the pump 132 can shift from operating in a default communication mode to operating in a mode prioritizing communicating with the mobile device 134 (and deprioritizing communicating with the CGM 136). The same is true for the host device 102 of the first system 100. Like the ambulatory infusion pump 132, it too can be configured to operate in different communications modes in order to improve communication with its peripheral devices 104 and 106.

Like FIG. 1B, FIG. 1C illustrates a system 160 that includes an ambulatory infusion pump 162, a mobile device 164, and a CGM 166. However, in this system 160, the mobile device 164 acts as host device, with the pump 162 and CGM 166 as peripherals connected thereto. Accordingly, in the third system 160, the mobile device 164 is configured to communicate with the pump 162 and CGM 166 via an antenna 168 of the mobile device 164. Like the host device 102 and the pump 132 of the second system 130, the mobile device 164 of this system 160 is can only communicate with a single peripheral device (e.g., pump 162, CGM 166) at a time. Accordingly, the mobile device 164 is unable to receive data from the CGM 166 while also communicating with the pump 162, and vice versa.

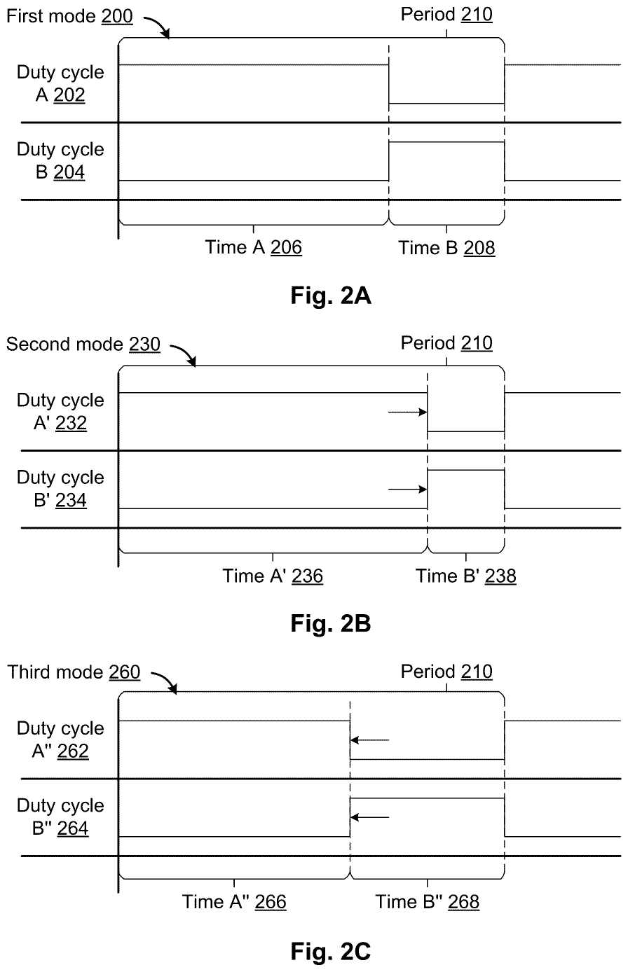

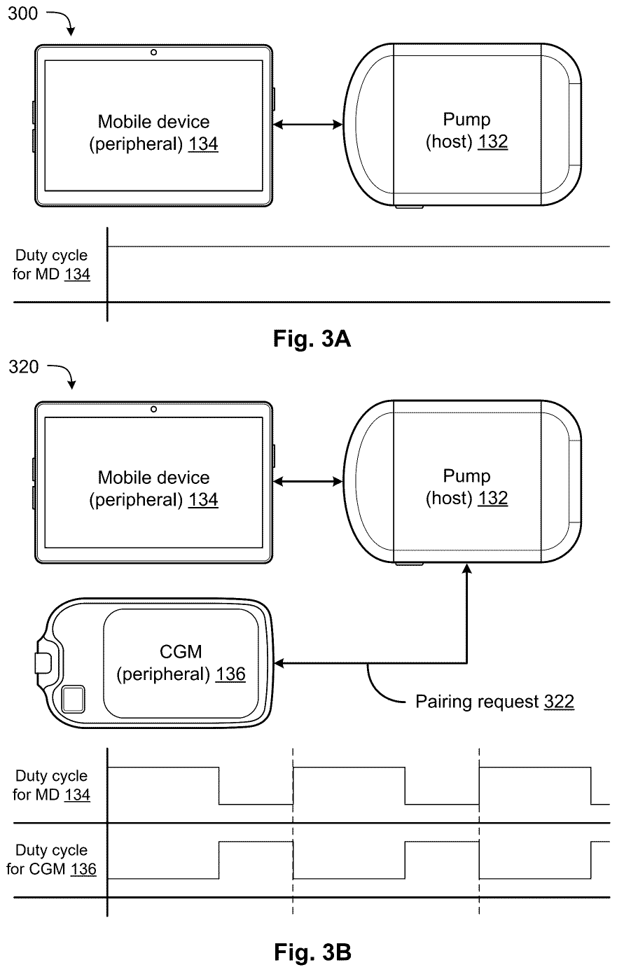

FIGS. 2A through 2C illustrate example communication modes (also referred to herein as “states”) 200, 230, and 260 and corresponding duty cycles 202, 204, 232, 234, 262, and 264 for communication between a host device (e.g., host 102, pump 132, mobile device 164) and multiple peripheral devices (e.g., peripherals 104 and 106, mobile device 134 and CGM 136, or pump 162 and CGM 166), according to various aspects of the subject technology. These Figures highlight how, in some embodiments, communication duty cycles can change between peripheral devices to accommodate the needs of the peripheral and host devices depending on what functions the peripheral or host devices are performing.

Note, the communication modes (or communication states) discussed herein are provided for illustrative purposes and are not intended to restrict the subject technology. Rather, they serve to highlight various ways in which a host device can adjust the amount of time it spends during a given period of time in communicating with its various peripheral devices. Although the following description provides concrete metrics with respect to communication modes and their respective duty cycles, a host device (e.g., an ambulatory infusion pump, a mobile device) can allocate different amounts of time or make different adjustments than those illustrated and discussed hereinbelow.

In this example, in the first communication mode 200, the host device communicates with a first peripheral device according to a first duty cycle 202 and a second peripheral device according to a second duty cycle 204. The first duty cycle is approximately a 70% duty cycle, which means that—for a given period of time 210 (e.g., 50, 100, 1000 milliseconds)—the host device spends 70% of that time 210 communicating with the first peripheral device and the remaining 30% communicating with the second peripheral device. It is understood that the exemplary duty cycles shown in FIGS. 2A-2C are merely for exemplary and illustrative purposes, and other duty cycle percentages for the peripherals could be used.

In other words, while operating in the first communication mode 200, the host device spends a first amount of time 206 (e.g., 70 ms) during each time period 210 (e.g., 100 ms) in communication with the first peripheral device. And the host device spends the remaining amount of time 208 (e.g., 3 ms) communicating with the second peripheral device. It is noted that, in embodiments where one of the peripheral devices (e.g., a CGM) collects data (e.g., blood glucose data) at regular intervals (e.g., every 5 mins), the time period 210 can be set based on the length of said intervals (e.g., 5 or 10 mins).

Although FIG. 2A suggests that the host device communicates first with the first peripheral device and then with the second peripheral device thereafter, this is not necessary. Operating according to a particular duty cycle requires only that the host device communicate with the corresponding peripheral device for a particular portion (e.g., 70%) of the time period, as defined by the communication mode and its respective duty cycle. This is true for the other communications modes discussed herein (incl. second and third modes 230 and 260), as well.

The second communication mode 230 of FIG. 2B differs from the first mode 200 with an increased first duty cycle 232 and a decreased second duty cycle 234. While operating in the second communication mode 230, the host device will spend more time 236 communicating with the first peripheral device than while operating in the first communication mode (compare times 206 and 236). Likewise, the host device will spend less time 238 communicating with the second peripheral device than while operating in the first communication mode 200 (compare times 208 and 238).

The host device may switch from operating in the first mode 200 to operating in the second mode 230 after determining that it needs to prioritize communication with the first peripheral device and/or deprioritize communication with the second peripheral device. As an example, the host device may operate in the first mode 200 while establishing an initial connection with the second peripheral device (e.g., Bluetooth pairing). Maintaining the connection with the second peripheral device may not require as much time as establishing it. Accordingly, after the initial connection is established, the host device can shift to operating in the second mode 230.

By contrast, the third communication mode 260 of FIG. 2C differs from the first mode 200 with a decreased first duty cycle 262 and an increased second duty cycle 264. While operating in the third communication mode 260, the host device will spend less time 266 communicating with the first peripheral device than when operating in the first communication mode (compare times 206 and 266). And the host device will spend more time 268 communicating with the second peripheral device than while operating in the first communication mode 200 (compare times 208 and 268).

The host device may switch from operating in the first mode 200 to operating in the third mode 260 after determining that it needs to prioritize communication with the second peripheral device and/or deprioritize communication with the first peripheral device. As an example, the host device may operate in the first mode 200 under normal circumstances and then switch to operating in the third mode 260 after receiving a request from the second peripheral device for a coordinated operation requiring increased communication with the third peripheral device (e.g., a bolus administration, a software update). In some embodiments, the host device may store a “priority data transmission event table” listing data transmission events for each peripheral. The data transmission events (e.g., pairing, bolus delivery, software update) may be associated with a ranking and/or a proposed duty cycle requirement for properly transmitting the data. The ranking and/or proposed duty cycle may be used as criteria by the host device to determine how to appropriately allocate duty cycles for each of the peripheral devices.

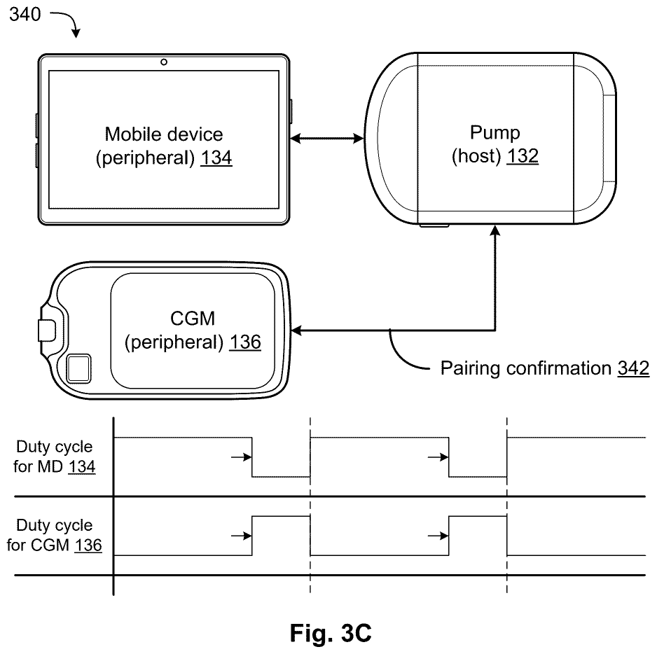

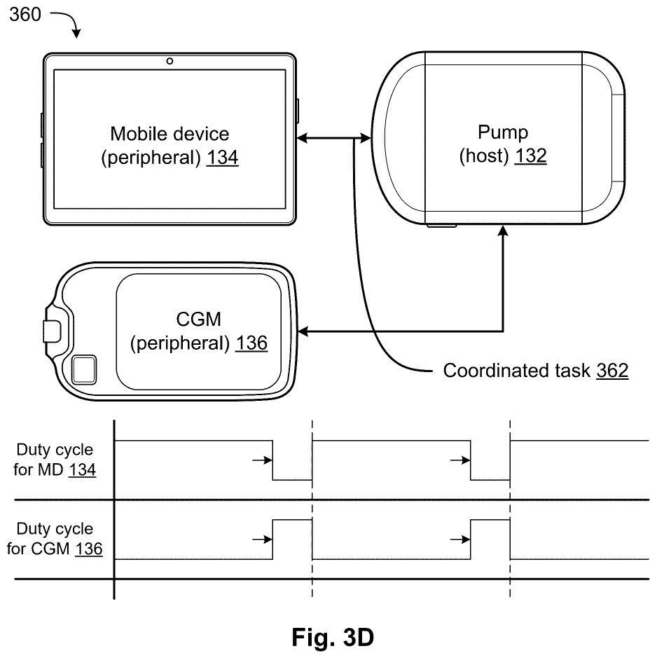

FIGS. 3A through 3D illustrate example communication scenarios 300, 320, 340, and 360 involving an ambulatory infusion pump 132 and peripheral devices (incl., a mobile device 134, a CGM 136) connected thereto, according to various aspects of the subject technology. In the scenario 300 of FIG. 3A, the pump 132 communicates with the mobile device 134 according to a continuous duty cycle (e.g., a 100% duty cycle) before establishing a connection with the CGM 136.

Thereafter, in the scenario 320 of FIG. 3B, the pump 132 receives a request 322 for pairing with the CGM 136 and shifts to operating in a communication mode that dedicates approximately 40% of its time to pairing with the CGM 136 and 60% of its time to communicating with the mobile device 134.

As depicted in the scenario 340 of FIG. 3C, after successfully pairing with the CGM 136 (see pairing confirmation 342), the pump 132 switches to operating in another communication mode. This shift reduces the amount of time the pump 132 spends communicating with the CGM 136 by approximately 10%, which accounts for the pump 132 needing less time to maintain a connection with the CGM 136 than to pair with it. While operating in this mode and communicating with the CGM 136, the pump 132 can receive blood-glucose data for a patient; though, the pump 132 may not receive blood-glucose data from the CGM 136 every the duty cycle—depending on how frequently the CGM 136 collects said data.

While operating in this communication mode, the pump 132 spends the remaining amount of time in each period communicating with the mobile device 134 (e.g., approximately 30%). Generally, communications with the mobile device 134 are more intensive than communications between the pump 132 and the CGM 136. Accordingly, the duty cycle associated with the mobile device 134 is greater than that associated with the CGM 136.

Occasionally, the pump 132 will receive a request from the mobile device 134 for execution of a coordinated operation 362, such as a bolus delivery or a software update. This is depicted in the scenario 360 of FIG. 3D. After receiving such a request, the pump 132 can switch to operating in another communication mode, which reduces the amount of time spent communicating with the CGM 136 in order to increase the amount of time spent communicating with the mobile device 134. This allows the pump 132 to prioritize completion of the coordinated operation while still communicating regularly with the CGM 136 in order to, for instance, maintain its connection with the CGM 136 or receive blood-glucose data therefrom. After completion of the operation, the pump 132 can return to operating in the earlier communication mode (e.g., depicted in FIG. 3C).

Although it is not depicted in the figures, the pump 132 may also receive coordinated operation requests from the CGM 136 and then adjust the duty cycle accordingly. For instance, after the pump first connects or re-connects to the CGM 136, the CGM 136 may initiate a data log transfer with the pump 132 to transfer all CGM data collected while the two devices were not connected to each other. In some embodiments, responsive to receiving a coordinated operation request from the CGM 136, the pump 132 increases the duty cycle for communicating with the CGM 136 and reduces the mobile device 134 duty cycle (e.g., in order to reduce the amount of time needed to complete a data log transfer).

Other data transmission events that can qualify as coordinated operations include (i) data transfers involved in priming the tubing and/or cannula of the pump 132, where the user may be interacting with the mobile device 134 during the priming process; (ii) data transfers involving CGM information, where CGM 136 sensors are scanned by the mobile device 134 (e.g., using near-field communication) and information (e.g., connection details, sensor information) is then transferred to the mobile device 134 and/or pump 132; (iii) data transfers involving pump 132 status, where the pump 132 communicates its status to the mobile device 134 (e.g., for display at the mobile device 134); and (iv) data transfers involving the mobile device 134 controlling the pump 132, for instance, by changing pump 132 settings and/or initiating therapy changes (e.g., adjusting the basal rate of the pump 132, shifting the pump 132 to an exercise mode). It is noted that coordinated operations can also occur between the mobile device 134 and the CGM 136, for instance, when the mobile device 134 is acting as a host device and the CGM 136 is a peripheral device thereto (see system 160 of FIG. 1C).

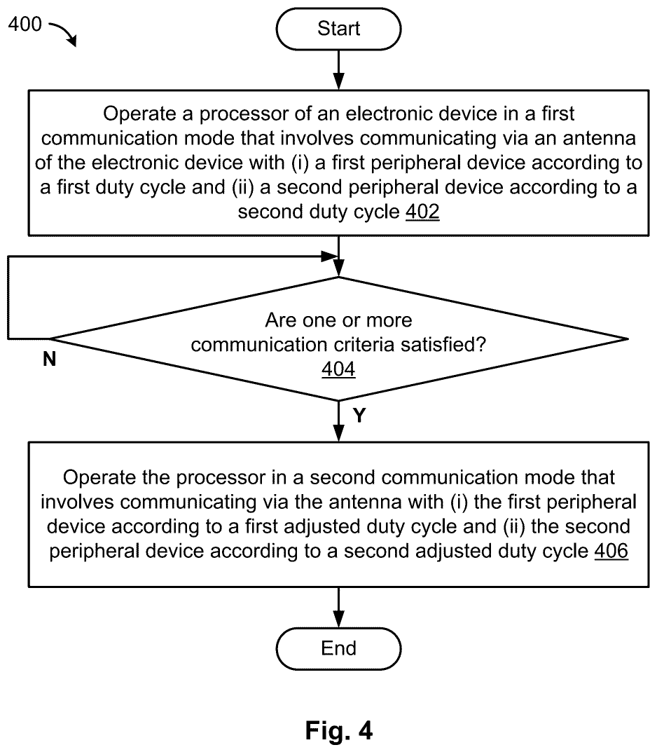

FIG. 4 illustrates an example process 400 for managing time division for single-antenna communications between a host device (e.g., host device 102, pump 132, mobile device 164) and multiple peripheral devices (e.g., peripheral devices 104 and 106, mobile device 134 and CGM 136, pump 162 and CGM 166), according to various aspects of the subject technology. The operations of the process 400 can be executed by the host devices discussed above with respect to FIGS. 1A through 3, such as the host device 102, ambulatory infusion pump 132, or mobile device 164. The operations can also be executed at least in part by a server connected to a host device or peripheral devices connected to the host device, such as the peripherals 104 and 106, the pump 162, the mobile device 134, or the CGM 136 or 166. Moreover, in some embodiments, the process 400 is executed by a processor configured to execute instructions stored in a non-transitory, computer-readable medium, where the instructions correspond to the various operations of said process 400.

The process 400 includes operating (402) a processor of an electronic device (e.g., host 102, pump 132, mobile device 164) in a first communication mode (e.g., mode 200). Operating in the first communication mode involves communicating via an antenna (e.g., antenna 108, 138 or 168) with a first peripheral device (e.g., peripheral 104, mobile device 134, pump 162) according to a first duty cycle (e.g., duty cycle 202) and a second peripheral device (e.g., peripheral 106, CGM 136 or 166) according to a second duty cycle (e.g., duty cycle 204).

The process 400 further includes determining (404) whether a communication criterion or multiple communication criteria are satisfied. As used herein, “communication criterion” refers to any standard or principle relevant to communication between the host device and one or more of the peripheral devices. For instance, a communication criterion may refer to “the first peripheral device is requesting to pair to the host device” or “the second peripheral device is requesting execution of a coordinated operation with the host device.”

After determining that one or more communication criteria are satisfied, the process 400 includes operating (406) the processor in a second communication mode (e.g., mode 230 or 260). Operating in the second communication mode involves communicating via the antenna with the first peripheral device according to a first adjusted duty cycle (e.g., duty cycle 232 or 262) and the second peripheral device according to a second adjusted duty cycle (e.g., duty cycle 243 or 264).

The first and second communication modes can be tailored, respectively, to a first state where the host device is establishing a connection with a peripheral device and a second state where the host device is connected to the peripheral device. Generally, the host device needs more time to establish the connection with the peripheral than to maintain the connection. Accordingly, the second communication mode may devote less time to communicating with the peripheral device relative to the first communication mode.

For instance, in some embodiments, determining that the one or more communication criteria are satisfied is based on detecting that the second peripheral device is successfully paired with the electronic device. In such embodiments, the first adjusted duty cycle may be greater than the first duty cycle, and the second adjusted duty cycle may be less than the second duty cycle.

In some of these embodiments, the process 400 further includes prior to operating in the first communication mode, operating the processor in a pre-pairing mode that involves communicating via the antenna with the first peripheral device according to a continuous duty cycle (e.g., a 100% duty cycle), receiving a request to pair with the second peripheral device, and, responsive to receiving the pairing request, switching from operating the processor in the pre-pairing mode to operating the processor in the first communication mode.

Additionally, the first and second communication modes can be tailored, respectively, to a first state where the host device is communicating regularly with the first and second peripheral devices and a second state where the host device is executing a coordinated operation with one of the peripheral devices. The coordinated operation is an operation that requires an increased amount of communication with one of the peripheral devices, such as a software update downloaded from a peripheral device to the host device, or a bolus administered by the host device based on parameters received from a peripheral device.

For instance, in some embodiments, determining that the one or more communication criteria are satisfied is based on receiving a request for a coordinated operation from the first peripheral device, where the coordinated operation requires increased communication with the first peripheral device. In such embodiments, the first adjusted duty cycle may be greater than the first duty cycle and the second adjusted duty cycle may be less than the second duty cycle. Additionally, in such embodiments, the process 400 may further include switching from operating the processor in the second communication mode to operating the processor in the first communication mode after completion of the coordinated operation.

In some of these embodiments, the coordinated operation includes administering a bolus of a medicament based on one or more parameters received from the first peripheral device. Alternatively, or additionally, in some of these embodiments, the coordinated operation includes installing a software update based on data received from the first peripheral device.

The host device can be configured to communicate with the peripheral devices based on how often one or both of the peripherals collects data. If one of the peripheral devices is a CGM (e.g., CGM 136 or 166) that collects blood glucose data at regular intervals, for example, then the host device may ensure it communicates at least one per said interval.

For instance, in some embodiments, the second peripheral device is configured to collect data at a predetermined frequency (e.g., once every 5 mins) with a corresponding period (e.g., 5 mins). Accordingly, in such embodiments, operating in the second communication mode may involve communicating with the first and second peripheral devices according to the period corresponding to the predetermined frequency.

In some of these embodiments, determining that the one or more communication criteria are satisfied is based on receiving the data (e.g., blood-glucose data) from the second peripheral device and determining that a rate of change of the data satisfies a predetermined threshold. In such embodiments, the first adjusted duty cycle is less than the first duty cycle, and the second adjusted duty cycle is greater than the second duty cycle. In this manner, the second communication mode can be tailored to prioritize the second peripheral device if the data collected thereby is changing at a particular rate.

Illustrative Clauses. For further reference, example aspects of the present disclosure are included below as numbered clauses. These clauses are provided for illustrative purposes and are not intended to limit the subject technology.
Clause 1. An electronic device for insulin delivery management, the electronic device comprising: an antenna; and a processor configured to: operate in a first communication mode that involves communicating via the antenna with (i) a first peripheral device according to a first duty cycle and (ii) a second peripheral device according to a second duty cycle; while operating in the first communication mode, determine that one or more communication criteria are satisfied; and responsive to determining that the one or more communication criteria are satisfied, switch from operating in the first communication mode to operating in a second communication mode that involves communicating via the antenna with (i) the first peripheral device according to a first adjusted duty cycle different from the first duty cycle and (ii) the second peripheral device according to a second adjusted duty cycle different from the second duty cycle.
Clause 2. The electronic device of Clause 1, wherein: determining that the one or more communication criteria are satisfied is based on detecting that the second peripheral device is successfully paired with the electronic device; the first adjusted duty cycle is greater than the first duty cycle; and the second adjusted duty cycle is less than the second duty cycle.
Clause 3. The electronic device of Clause 2, wherein the processor is further configured to, prior to operating in the first communication mode: operate in a pre-pairing mode that involves communicating via the antenna with the first peripheral device according to a continuous duty cycle; receive a request to pair with the second peripheral device; and responsive to receiving the pairing request, switch from operating in the pre-pairing mode to operating in the first communication mode.
Clause 4. The electronic device of any one of Clauses 1-3, wherein: determining that the one or more communication criteria are satisfied is based on receiving a request for a coordinated operation from the first peripheral device, wherein the coordinated operation requires increased communication with the first peripheral device; the first adjusted duty cycle is greater than the first duty cycle and the second adjusted duty cycle is less than the second duty cycle; and the processor is further configured to switch from operating in the second communication mode to operating in the first communication mode after completion of the coordinated operation.
Clause 5. The electronic device of Clause 4, wherein the coordinated operation comprises administering a bolus of a medicament based on one or more parameters received from the first peripheral device.
Clause 6. The electronic device of either Clause 4 or 5, wherein the coordinated operation comprises installing a software update based on data received from the first peripheral device.
Clause 7. The electronic device of any one of Clauses 1-6, wherein: the second peripheral device is configured to collect data at a predetermined frequency with a corresponding period; and operating in the second communication mode further involves communicating with the first and second peripheral devices according to the period corresponding to the predetermined frequency.
Clause 8. The electronic device of Clause 7, wherein: determining that the one or more communication criteria are satisfied is based on (i) receiving the data from the second peripheral device and (ii) determining that a rate of change of the data satisfies a predetermined threshold; the first adjusted duty cycle is less than the first duty cycle; and the second adjusted duty cycle is greater than the second duty cycle.
Clause 9. The electronic device of any one of Clauses 1-8, wherein: the electronic device is an ambulatory infusion pump; the first peripheral device is a mobile device; and the second peripheral device is a CGM.
Clause 10. The electronic device of any one of Clauses 1-9, wherein: the electronic device is a mobile device; the first peripheral device is an ambulatory infusion pump; and the second peripheral device is a CGM.
Clause 11. A computer-implemented method of managing time division for single-antenna communications regarding insulin delivery management, the method comprising: operating a processor of an electronic device in a first communication mode that involves communicating via an antenna of the electronic device with (i) a first peripheral device according to a first duty cycle and (ii) a second peripheral device according to a second duty cycle; while operating the processor in the first communication mode, determining that one or more communication criteria are satisfied; and responsive to determining that the one or more communication criteria are satisfied, switching from operating the processor in the first communication mode to operating the processor in a second communication mode that involves communicating via the antenna with (i) the first peripheral device according to a first adjusted duty cycle different from the first duty cycle and (ii) the second peripheral device according to a second adjusted duty cycle different from the second duty cycle.
Clause 12. The computer-implemented method of Clause 11, wherein: determining that the one or more communication criteria are satisfied is based on detecting that the second peripheral device is successfully paired with the electronic device; the first adjusted duty cycle is greater than the first duty cycle; and the second adjusted duty cycle is less than the second duty cycle.
Clause 13. The computer-implemented method of Clause 12, further comprising, prior to operating in the first communication mode: operating the processor in a pre-pairing mode that involves communicating via the antenna with the first peripheral device according to a continuous duty cycle; receiving a request to pair with the second peripheral device; and responsive to receiving the pairing request, switching from operating the processor in the pre-pairing mode to operating the processor in the first communication mode.
Clause 14. The computer-implemented method of any one of Clauses 11-13, wherein: determining that the one or more communication criteria are satisfied is based on receiving a request for a coordinated operation from the first peripheral device, wherein the coordinated operation requires increased communication with the first peripheral device; the first adjusted duty cycle is greater than the first duty cycle and the second adjusted duty cycle is less than the second duty cycle; and the method further comprises switching from operating the processor in the second communication mode to operating the processor in the first communication mode after completion of the coordinated operation.
Clause 15. The computer-implemented method of Clause 14, wherein the coordinated operation comprises administering a bolus of a medicament based on one or more parameters received from the first peripheral device.
Clause 16. The computer-implemented method of either Clause 14 or 15, wherein the coordinated operation comprises installing a software update based on data received from the first peripheral device.
Clause 17. The computer-implemented method of any one of Clauses 11-16, wherein: the second peripheral device is configured to collect data at a predetermined frequency with a corresponding period; and operating in the second communication mode further involves communicating with the first and second peripheral devices according to the period corresponding to the predetermined frequency.
Clause 18. The computer-implemented method of Clause 17, wherein: determining that the one or more communication criteria are satisfied is based on (i) receiving the data from the second peripheral device and (ii) determining that a rate of change of the data satisfies a predetermined threshold; the first adjusted duty cycle is less than the first duty cycle; and the second adjusted duty cycle is greater than the second duty cycle.
Clause 19. The computer-implemented method of any one of Clauses 11-18, wherein: the electronic device is an ambulatory infusion pump; the first peripheral device is a mobile device; and the second peripheral device is a CGM.
Clause 20. The computer-implemented method of any one of Clauses 11-19, wherein: the electronic device is a mobile device; the first peripheral device is an ambulatory infusion pump; and the second peripheral device is a CGM.
Clause 21. A non-transitory, computer-readable medium including instructions that, when executed by a processor of an electronic device for insulin delivery management, cause the electronic device to: operate in a first communication mode that involves communicating via an antenna of the electronic device with (i) a first peripheral device according to a first duty cycle and (ii) a second peripheral device according to a second duty cycle; while operating in the first communication mode, determine that one or more communication criteria are satisfied; and responsive to determining that the one or more communication criteria are satisfied, switch from operating in the first communication mode to operating in a second communication mode that involves communicating via the antenna with (i) the first peripheral device according to a first adjusted duty cycle different from the first duty cycle and (ii) the second peripheral device according to a second adjusted duty cycle different from the second duty cycle.
Clause 22. The non-transitory, computer-readable medium of Clause 21, wherein: determining that the one or more communication criteria are satisfied is based on detecting that the second peripheral device is successfully paired with the electronic device; the first adjusted duty cycle is greater than the first duty cycle; and the second adjusted duty cycle is less than the second duty cycle.
Clause 23. The non-transitory, computer-readable medium of Clause 22, wherein the instructions further cause the electronic device to, prior to operating in the first communication mode: operate in a pre-pairing mode that involves communicating via the antenna with the first peripheral device according to a continuous duty cycle; receive a request to pair with the second peripheral device; and responsive to receiving the pairing request, switch from operating in the pre-pairing mode to operating in the first communication mode.
Clause 24. The non-transitory, computer-readable medium of any one of Clauses 21-23, wherein: determining that the one or more communication criteria are satisfied is based on receiving a request for a coordinated operation from the first peripheral device, wherein the coordinated operation requires increased communication with the first peripheral device; the first adjusted duty cycle is greater than the first duty cycle and the second adjusted duty cycle is less than the second duty cycle; and the instructions further cause the electronic device to switch from operating in the second communication mode to operating in the first communication mode after completion of the coordinated operation.
Clause 25. The non-transitory, computer-readable medium of Clause 24, wherein the coordinated operation comprises administering a bolus of a medicament based on one or more parameters received from the first peripheral device.
Clause 26. The non-transitory, computer-readable medium of either Clause 24 or 25, wherein the coordinated operation comprises installing a software update based on data received from the first peripheral device.
Clause 27. The non-transitory, computer-readable medium of any one of Clauses 21-26, wherein: the second peripheral device is configured to collect data at a predetermined frequency with a corresponding period; and operating in the second communication mode further involves communicating with the first and second peripheral devices according to the period corresponding to the predetermined frequency.
Clause 28. The non-transitory, computer-readable medium of Clause 27, wherein: determining that the one or more communication criteria are satisfied is based on (i) receiving the data from the second peripheral device and (ii) determining that a rate of change of the data satisfies a predetermined threshold; the first adjusted duty cycle is less than the first duty cycle; and the second adjusted duty cycle is greater than the second duty cycle.
Clause 29. The non-transitory, computer-readable medium of any one of Clauses 21-28, wherein: the electronic device is an ambulatory infusion pump; the first peripheral device is a mobile device; and the second peripheral device is a CGM.
Clause 30. The non-transitory, computer-readable medium of any one of Clauses 21-29, wherein: the electronic device is a mobile device; the first peripheral device is an ambulatory infusion pump; and the second peripheral device is a CGM.
Clause 31. A system for insulin delivery management comprising: a first peripheral device; a second peripheral device; and an electronic device comprising: an antenna; and a processor configured to: operate in a first communication mode that involves communicating via the antenna with (i) the first peripheral device according to a first duty cycle and (ii) the second peripheral device according to a second duty cycle; while operating in the first communication mode, determine that one or more communication criteria are satisfied; and responsive to determining that the one or more communication criteria are satisfied, switch from operating in the first communication mode to operating in a second communication mode that involves communicating via the antenna with (i) the first peripheral device according to a first adjusted duty cycle different from the first duty cycle and (ii) the second peripheral device according to a second adjusted duty cycle different from the second duty cycle.
Clause 32. The system of Clause 31, wherein: determining that the one or more communication criteria are satisfied is based on detecting that the second peripheral device is successfully paired with the electronic device; the first adjusted duty cycle is greater than the first duty cycle; and the second adjusted duty cycle is less than the second duty cycle.
Clause 33. The system of Clause 32, wherein the processor of the electronic device is further configured to, prior to operating in the first communication mode: operate in a pre-pairing mode that involves communicating via the antenna with the first peripheral device according to a continuous duty cycle; receive a request to pair with the second peripheral device; and responsive to receiving the pairing request, switch from operating in the pre-pairing mode to operating in the first communication mode.
Clause 34. The system of any one of Clauses 31-33, wherein: determining that the one or more communication criteria are satisfied is based on receiving a request for a coordinated operation from the first peripheral device, wherein the coordinated operation requires increased communication with the first peripheral device; the first adjusted duty cycle is greater than the first duty cycle and the second adjusted duty cycle is less than the second duty cycle; and the processor of the electronic device is further configured to switch from operating in the second communication mode to operating in the first communication mode after completion of the coordinated operation.
Clause 35. The system of Clause 34, wherein the coordinated operation comprises administering a bolus of a medicament based on one or more parameters received from the first peripheral device.
Clause 36. The electronic device of either Clause 34 or 35, wherein the coordinated operation comprises installing a software update based on data received from the first peripheral device.
Clause 37. The system of any one of Clauses 31-36, wherein: the second peripheral device is configured to collect data at a predetermined frequency with a corresponding period; and operating in the second communication mode further involves communicating with the first and second peripheral devices according to the period corresponding to the predetermined frequency.
Clause 38. The system of Clause 37, wherein: determining that the one or more communication criteria are satisfied is based on (i) receiving the data from the second peripheral device and (ii) determining that a rate of change of the data satisfies a predetermined threshold; the first adjusted duty cycle is less than the first duty cycle; and the second adjusted duty cycle is greater than the second duty cycle.
Clause 39. The system of any one of Clauses 31-38, wherein: the electronic device is an ambulatory infusion pump; the first peripheral device is a mobile device; and the second peripheral device is a CGM.
Clause 40. The system of any one of Clauses 31-39, wherein: the electronic device is a mobile device; the first peripheral device is an ambulatory infusion pump; and the second peripheral device is a CGM.

Further Consideration. The specific order or hierarchy of steps in the processes disclosed herein is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Headings and subheadings, if any, are used for convenience only and do not limit the invention described herein.

The predicate words “configured to,” “operable to,” and “programmed to” do not imply any particular tangible or intangible modification of a subject, but rather are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as “implementations” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein, the terms “determine” and “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the term “message” encompasses a wide variety of formats for communicating (e.g., transmitting or receiving) information. A message may include a machine readable aggregation of information such as an XML document, fixed field message, comma separated message, JSON, a custom protocol, or the like. A message may, in some embodiments, include a signal utilized to transmit one or more representations of the information. While recited in the singular, it will be appreciated that a message may be composed, transmitted, stored, received, and so on in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some embodiments, an n-input switch may be included to provide selective functionality where n is the number of inputs used to make the selection.

As used herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine-learning assessment model, or combinations thereof.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

What is claimed is:

1. An electronic device for insulin delivery management, the electronic device comprising:
an antenna; and
a processor configured to:
operate in a first communication mode that involves communicating via the antenna with (i) a first peripheral device according to a first duty cycle and (ii) a second peripheral device according to a second duty cycle;
while operating in the first communication mode, determine that one or more communication criteria are satisfied; and
responsive to determining that the one or more communication criteria are satisfied, switch from operating in the first communication mode to operating in a second communication mode that involves communicating via the antenna with (i) the first peripheral device according to a first adjusted duty cycle different from the first duty cycle and (ii) the second peripheral device according to a second adjusted duty cycle different from the second duty cycle.

2. The electronic device of claim 1, wherein:
determining that the one or more communication criteria are satisfied is based on detecting that the second peripheral device is successfully paired with the electronic device;
the first adjusted duty cycle is greater than the first duty cycle; and
the second adjusted duty cycle is less than the second duty cycle.

3. The electronic device of claim 2, wherein the processor is further configured to, prior to operating in the first communication mode:
operate in a pre-pairing mode that involves communicating via the antenna with the first peripheral device according to a continuous duty cycle;
receive a request to pair with the second peripheral device; and
responsive to receiving the pairing request, switch from operating in the pre-pairing mode to operating in the first communication mode.

4. The electronic device of claim 1, wherein:
determining that the one or more communication criteria are satisfied is based on receiving a request for a coordinated operation from the first peripheral device, wherein the coordinated operation requires increased communication with the first peripheral device;
the first adjusted duty cycle is greater than the first duty cycle and the second adjusted duty cycle is less than the second duty cycle; and
the processor is further configured to switch from operating in the second communication mode to operating in the first communication mode after completion of the coordinated operation.

5. The electronic device of claim 4, wherein the coordinated operation comprises administering a bolus of a medicament based on one or more parameters received from the first peripheral device.

6. The electronic device of claim 4, wherein the coordinated operation comprises installing a software update based on data received from the first peripheral device.

7. The electronic device of claim 1, wherein:
the second peripheral device is configured to collect data at a predetermined frequency with a corresponding period; and
operating in the second communication mode further involves communicating with the first and second peripheral devices according to the period corresponding to the predetermined frequency.

8. The electronic device of claim 7, wherein:
determining that the one or more communication criteria are satisfied is based on (i) receiving the data from the second peripheral device and (ii) determining that a rate of change of the data satisfies a predetermined threshold;
the first adjusted duty cycle is less than the first duty cycle; and
the second adjusted duty cycle is greater than the second duty cycle.

9. The electronic device of claim 1, wherein:
the electronic device is an ambulatory infusion pump;
the first peripheral device is a mobile device; and
the second peripheral device is a CGM.

10. The electronic device of claim 1, wherein:
the electronic device is a mobile device;
the first peripheral device is an ambulatory infusion pump; and
the second peripheral device is a CGM.

11. A computer-implemented method of managing time division for single-antenna communications regarding insulin delivery management, the method comprising:
operating a processor of an electronic device in a first communication mode that involves communicating via an antenna of the electronic device with (i) a first peripheral device according to a first duty cycle and (ii) a second peripheral device according to a second duty cycle;
while operating the processor in the first communication mode, determining that one or more communication criteria are satisfied; and
responsive to determining that the one or more communication criteria are satisfied, switching from operating the processor in the first communication mode to operating the processor in a second communication mode that involves communicating via the antenna with (i) the first peripheral device according to a first adjusted duty cycle different from the first duty cycle and (ii) the second peripheral device according to a second adjusted duty cycle different from the second duty cycle.

12. The computer-implemented method of claim 11, wherein:
determining that the one or more communication criteria are satisfied is based on detecting that the second peripheral device is successfully paired with the electronic device;
the first adjusted duty cycle is greater than the first duty cycle; and
the second adjusted duty cycle is less than the second duty cycle.

13. The computer-implemented method of claim 12, further comprising, prior to operating in the first communication mode:
operating the processor in a pre-pairing mode that involves communicating via the antenna with the first peripheral device according to a continuous duty cycle;
receiving a request to pair with the second peripheral device; and
responsive to receiving the pairing request, switching from operating the processor in the pre-pairing mode to operating the processor in the first communication mode.

14. The computer-implemented method of claim 11, wherein:
determining that the one or more communication criteria are satisfied is based on receiving a request for a coordinated operation from the first peripheral device, wherein the coordinated operation requires increased communication with the first peripheral device;
the first adjusted duty cycle is greater than the first duty cycle and the second adjusted duty cycle is less than the second duty cycle; and
the method further comprises switching from operating the processor in the second communication mode to operating the processor in the first communication mode after completion of the coordinated operation.

15. The computer-implemented method of claim 14, wherein the coordinated operation comprises administering a bolus of a medicament based on one or more parameters received from the first peripheral device.

16. The computer-implemented method of claim 14, wherein the coordinated operation comprises installing a software update based on data received from the first peripheral device.

17. The computer-implemented method of claim 11, wherein:
the second peripheral device is configured to collect data at a predetermined frequency with a corresponding period; and
operating in the second communication mode further involves communicating with the first and second peripheral devices according to the period corresponding to the predetermined frequency.

18. The computer-implemented method of claim 17, wherein:
determining that the one or more communication criteria are satisfied is based on (i) receiving the data from the second peripheral device and (ii) determining that a rate of change of the data satisfies a predetermined threshold;
the first adjusted duty cycle is less than the first duty cycle; and
the second adjusted duty cycle is greater than the second duty cycle.

19. The computer-implemented method of claim 11, wherein:
the electronic device is an ambulatory infusion pump;
the first peripheral device is a mobile device; and
the second peripheral device is a CGM.

20. A non-transitory, computer-readable medium including instructions that, when executed by a processor of an electronic device for insulin delivery management, cause the electronic device to:
operate in a first communication mode that involves communicating via an antenna of the electronic device with (i) a first peripheral device according to a first duty cycle and (ii) a second peripheral device according to a second duty cycle;
while operating in the first communication mode, determine that one or more communication criteria are satisfied; and
responsive to determining that the one or more communication criteria are satisfied, switch from operating in the first communication mode to operating in a second communication mode that involves communicating via the antenna with (i) the first peripheral device according to a first adjusted duty cycle different from the first duty cycle and (ii) the second peripheral device according to a second adjusted duty cycle different from the second duty cycle.
